(19) United States
(12) Patent Application Publication
(10) Pub. No.: US 2004/0018210 A1
(43) Pub. Date: Jan. 29, 2004

(54) Cholesterol-lowering agent

(76) Inventors: Hassan Hajjaj, Fontaines St.-Martin (FR); Catherine Mace, Lutry (CH); Peter Niederberger, Epalinges (CH); Laurent-Bernard Fay, Evian (FR)

Correspondence Address: WINSTON & STRAWN, PATENT DEPARTMENT, 1400 L STREET, N.W., WASHINGTON, DC, 20005-3502, US

(21) Appl. No.: 10/629,823

(22) Filed: Jul. 30, 2003

Related U.S. Application Data
(63) Continuation of application No. PCT/EP02/00787, filed on Jan. 24, 2002.

(30) Foreign Application Priority Data
Jan. 31, 2001 (EP) 01102218.3

(51) Int. Cl. A61K 35/84

(52) U.S. Cl. 424/195.15

(57) ABSTRACT

A hypocholesterolaemic agent and a process for its preparation by extraction from edible fungi, along with methods of use of the agent for preventing the synthesis of cholesterol in a person. These methods include the administration of the agent alone or in a food or beverage. The foods or beverages that contain this agent are another feature of the invention.

### CROSS-REFERENCE TO RELATED APPLICATION

This application is a continuation of International application PCT/EP02/00787 filed Jan. 24, 2002, the entire content of which is expressly incorporated herein by reference thereto.

### BACKGROUND

The present invention relates to a hypocholesterolaemic agent obtained from edible fungi.

The nutritional considerations of consumers currently drive foodstuffs producers to propose foods having improved nutritional functionalities.

It is recognized that a high serum cholesterol level is an atherosclerosis risk factor which cannot be ignored. The reduction in this serum cholesterol level is thus a means of combating the risk of cardiovascular diseases. Given that the majority of the cholesterol in the circulation is the product of a de novo synthesis, the inhibition of endogenous cholesterol biosynthesis seems to be an interesting route for treatment or prevention of hypercholesterolaemia.

The biosynthesis of cholesterol comprises three main stages. The first, condensation, allows three acetyl-CoA to pass to mevalonate. This condensation involves the participation, inter alia, of 3-hydroxy-3-methylglutaryl-CoA reductase (HMGCoA reductase). The mevalonate formed in this way is then polymerized into squalene via polyisoprene intermediates. Finally, the squalene leads to cholesterol by cyclization/demethylation via the formation of reaction intermediates, inter alia lanosterol and its derivatives.

Various compounds are used to inhibit the synthesis of cholesterol. The statins in particular act very early on in the synthesis by inhibition of HMGCoA reductase. Lovastatin, which is isolated and purified from various fungi and/or moulds, has been used as a hypocholesterolaemic agent in the form of a medicament or nutritional complement. DE 4402591 thus describes the production and isolation of lovastatin and mevinolinic acid from Pleurotus ostreatus, and the use of these compounds for inhibition of cholesterol biosynthesis. However, the use of lovastatin requires an isolation and a purification before its use. On the other hand, the compounds of the class of statins which are inhibitors of HMGCoA reductase induce undesirable secondary effects. In fact, HMGCoA reductase is an enzyme which acts very early on in the cholesterol biosynthesis chain (transformation of HMGCoA into mevalonate), and its inhibition consequently tends to modify the synthesis of other compounds derived from mevalonate. Thus, by suppressing the production of mevalonate, a reduction in the synthesis of various steroids, hormones, coenzyme Q10, isopentenyl tRNA, isoprenoid derivatives or dolichol, for example, is observed. The depletion of such metabolites is responsible, inter alia, for hepatic and muscular disorders via an increase in the level of hepatic transaminases, for example (Hiyoshi et al., J. of Lipid Research, 2000, (41) 1136-1141).

From this point of view, compounds which are capable of inhibiting the terminal stages of the cholesterol synthesis chain have been sought in order to avoid the inherent problems of inhibitors of HMGCoA reductase. Various compounds of synthesis which are inhibitors of the terminal stages of the demethylation of lanosterol into 14-demethyl-lanosterol exist. The majority of these compounds are oxygenated derivatives of sterols.

However, all these inhibitors are pure products of chemical synthesis, such as those described in WO9113903. Thus, in addition to the fact that their manufacture passes through a long, complex and onerous chemical synthesis, these compounds fall in the class of medicaments, which involves clinical studies for homologation, registration and authorization.

Komoda et al. (Chem. Pharm. Bull., 1989 37(2) 531-533) describe oxygenated derivatives of lanosterol obtained by chemical modification of ganoderic acid isolated from Ganoderma lucidum. It is stated in this document that only derivatives modified by a chemical route have a hypocholesterolaemic action. Ganoderic acid B and its ester derivative are used to obtain various other derivatives which do not have inhibitory activity on the synthesis of cholesterol.

Various fungi are described for their hypocholesterolaemic properties. KR 9303886 thus describes the preparation of hypocholesterolaemic foods from medicinal fungi by drying, pulverization, extraction with boiling water and concentration. It seems that such extracts act at the HMGCoA reductase level (Bobek et al., Casopis Lebaru Ceskych, 1997 136(6) 186-190; Bobek et al., Experientia, 1995 51(6) 589-591). DE 4402591, mentioned above, also suggests that certain edible fungi are rich in HMGCoA reductase inhibitors. Despite these disclosures, there still is a need for hypocholesterolaemic agents which are not obtained by chemical synthesis and can be used in a food product so that, when ingested, they act on the terminal stages of the cholesterol biosynthesis chain. The present invention now satisfies this need.

### SUMMARY OF THE INVENTION

The present invention relates to a process for preparing a hypocholesterolaemic agent of an oxygenated natural derivative of lanosterol obtained from edible fungi. This process comprises:

steeping an edible fungus in a first solvent under temperature and time conditions effective to extract an active fraction in a liquid phase,

separating the liquid phase from solid materials,

obtaining a dry extract of the active fraction from the liquid phase,

forming an aqueous phase of the dry extract and water,

contacting the aqueous phase with a second solvent that has a lower polarity than the first solvent and that is immiscible with water, with the contacting conducted under conditions sufficient to extract the active fraction in an organic phase, separating the organic phase from the aqueous phase, and

obtaining as the hypocholesterolaemic agent the active fraction recovered from the organic phase.

In this process, the steeping is carried out for 4 to 96 hours at a temperature of between 5 and 30° C. Also the dry extract is preferably obtained by evaporating the liquid phase. The process further comprises adjusting the pH of the aqueous phase to a value of between 2 and 5 before the extraction with the second solvent. In addition, the contacting is preferably carried out by repeated washings of the aqueous phase.

The invention also relates to a hypocholesterolaemic agent obtainable by the process described herein. Such an agent is rich in oxygenated natural derivatives of lanosterol.

Another embodiment relates to an edible composition for inhibiting synthesis of cholesterol in a person comprising a food or beverage and the hypocholesterolaemic agent in an effective amount therein.

In yet another embodiment of the invention, methods of treatment are disclosed. One method for inhibiting synthesis of cholesterol comprises administering to a person in need of such treatment the hypocholesterolaemic agent in an effective amount thereof. Another method for inhibiting synthesis of cholesterol comprises administering to a person in need of such treatment a food or beverage containing the hypocholesterolaemic agent in an effective amount therein.

### BRIEF DESCRIPTION OF THE DRAWINGS

FIGS. 1 and 2 are NMR graphs of Ganoderol A by H1 and C13 analyses, respectively.

### DETAILED DESCRIPTION OF THE PREFERRED EMBODIMENTS

“Hypocholesterolaemic agent” is understood as meaning an agent which inhibits the synthesis of cholesterol, and more particularly by acting on the terminal stages of the cholesterol biosynthesis chain, due to its content of oxygenated natural derivatives of lanosterol.

“Natural derivatives” is understood as meaning compounds which have not been subjected to any chemical modification by any means whatsoever. They are oxygenated derivatives of lanosterol obtained directly from edible fungi.

In addition, “edible fungi” is understood as meaning fungi with a long tradition of consumption as food. These are thus understood as neither toxic nor poisonous fungi, which may or may not be distinguished by their gustative and/or aromatic qualities. They may be, for example, fungi chosen from the group consisting of the following classification orders: Agaricalles, Aphyllophorales and Stereales. Preferably, they can be fungi chosen from the group consisting of: Pleurotus eryngii, Pleurotus eous, Ganoderma lucidum, Grifola frondosa, Pleurotus ostreatus, Agrocybe aegerita, Pholiota nameko, Pleurotus citrinopileatus and Flamulina velutipes.

The invention also relates to the use of a hypocholesterolaemic agent comprising oxygenated natural derivatives of lanosterol obtained from edible fungi in a foodstuffs product.

Finally, the process for the preparation of the hypocholesterolaemic agent according to the present invention comprises the following stages:

steeping of an edible fungus in a first solvent,

separation of the solid and liquid phases,

evaporation of the liquid phase until a dry extract is obtained,

taking up of the dry extract in water,

extraction of the aqueous phase with the aid of a second solvent which has a lower polarity than the first solvent and is immiscible with water and

separation of the aqueous and organic phases.

The organic extract obtained in this way after removal of the aqueous phase is the hypocholesterolaemic agent rich in oxygenated natural derivatives of lanosterol and is used for the in vitro evaluations, the fractionations and purifications.

The steeping stage can be preceded by a grinding stage and is carried out with the fresh or dried fungus. This steeping can be carried out at ambient temperature or at a refrigeration temperature. Depending on the steeping temperature chosen, the latter will be carried out over a period of longer or shorter duration. The steeping stage can therefore extend over a period which can be between 6 and 72 hours. The first solvent used for this steeping can be methanol, ethanol or chloroform, for example, used by themselves or as a mixture.

The separation of the solid and liquid phases can be carried out by filtration over paper or over gauze, by centrifugation or by decanting, for example.

After evaporation of the liquid phase, preferably at a temperature close to ambient temperature, the dry extract obtained can be taken up with an amount of water of between 5 and 100% (v/v) with respect to the amount of the first solvent used for the steeping. In order to facilitate the extraction with the second solvent, the pH of this aqueous solution obtained in this way can be adjusted to an acid pH value, that is to say to a value of between 2 and 4, for example, before the extraction with the aid of the second solvent. This acidification can be carried out with the aid of hydrochloric acid, for example.

The aqueous solution obtained in this way can be subjected to an extraction with the aid of a second solvent which is immiscible with water and has a lower polarity than the first, such as ethyl acetate, isopropanol or chloroform, for example, volume for volume. The second solvent will be chosen according to these double criteria of lower polarity than the first solvent and immiscibility with water. In the particular case of the possible use of chloroform as the first solvent, for example, this will not constitute the second solvent and this second solvent will be chosen with a lower polarity than chloroform and immiscibility with water. Such an extraction can be carried out several times consecutively, for example. The organic phase can be collected by decanting, for example, and evaporated, preferably in vacuo at a temperature of the order of 25 to 35° C. Before this evaporation stage, the organic phase can be dried if appropriate, for example with the aid of anhydrous Na₂SO₄.

Thus, once the organic phase has been evaporated completely, an extract is obtained in a pulverulent form rich in oxygenated natural derivatives of lanosterol. Such an extract can be added to a foodstuffs product, for example.

In the present context, the hypocholesterolaemic effect of the present product is evaluated in a qualitative manner on the model of T9A4 human hepatic cells cultured in vitro. Although it is known that such results cannot be transferred directly to man in vivo, they nevertheless provide useful indications. The hypocholesterolaemic effect is evaluated by measurement of the de novo synthesis of cholesterol on human hepatic cells in culture by measuring the incorporation of C¹⁴-acetate. A means of evaluation of this de novo synthesis is described in a method presented below.

The agent obtained after extraction from the fungus can be purified, fractionated, analysed and characterized by liquid and gas chromatography, mass spectroscopy and nuclear magnetic resonance in order to identify the oxygenated derivatives of lanosterol contained therein. The materials and methods used for such a characterization are described in detail in the methods presented below.

In Vitro Analysis of the Hypocholesterolaemic Effect

T9A4 human hepatic cells are grown on LCM medium (Biofluids, Rockville, Md.) at 37° C. under 3.5% CO₂. The cells are seeded in 24-well culture boxes and incubated to confluence with 1 mM C14-acetate (1 mCi/mol, Amersham) for 20 hours in the absence (control) or in the presence of the extract rich in oxygenated derivatives of lanosterol obtained from fungi and/or purified fractions, dissolved in methanol.

A lipid extraction is carried out by incubation in a hexane:isopropanol (3:2) mixtures for 30 minutes at ambient temperature. The extract is then dried under nitrogen and then redissolved in hexane and subjected to thin layer high performance chromatography (Merck, Darmstadt, Germany) with a hexane:diethyl ether:acetic acid (75:25:1) mixture as the solvent. The cholesterol neosynthesis is determined by measuring the incorporation of C¹⁴-acetate within the cholesterol with the aid of an imager (Camberra Packard, Zurich, Switzerland) and is expressed in % of the control.

The results are shown in Table 1. ID50 represents the dose which inhibits the cholesterol synthesis by 50%. It can be seen that the various extracts have a considerable hypocholesterolaemic activity. Furthermore, the extracts obtained with Ganoderma lucidum, Pleurotus citrinopileatus and Flamulina velutipes show a very high activity with a 50% inhibition threshold of the order of one to less than 14 μg/ml.

Table 1: In vitro hypocholesterolaemic activity observed on human hepatic cells with the extracts obtained from edible fungi.

| Fungi | ID₅₀ (μ/ml) |
|---|---|
| Pleurotus eryngii | >150 |
| Pleurotus eous | >70 |
| Ganoderma lucidum | 1 |
| Grifola frondosa | 180 |
| Pleurotus ostreatus | 150 |
| Agrocybe aegerita | >150 |
| Pholiota nameko | >300 |
| Pleurotus citrinopileatus | 10 |
| Flamunila velutipes | <14 |

The active crude extracts from Ganoderma lucidum and Pleurotus citrinopileatus were purified and tested for their hypocholesterolaemic activities. The results are shown in table 2. It can be seen that the crude extract obtained from Ganoderma lucidum itself contains a large amount of hypocholesterolaemic compounds of which the dose which inhibits cholesterol biosynthesis to 50% is less than 3 μg/ml. The inhibition observed with the purified fractions from Pleurotus ostreatus have a higher but nevertheless particularly interesting ID50.

Physico-Chemical Characterization of Compounds Contained in the Fungi Extracts

Mass Spectrometry

HPLC/MS (High Performance Liquid Chromatography/Mass Spectrometry)

The analyses were carried out using either a Micromass AutoSpec OA-TOF mass spectrometer connected to a Waters 2690 HPLC system or a Finnigan TSQ-700 triple quadrupole mass spectrometer connected to a Waters HPLC system consisting of a 757 autosampler, a 600-MS pump and a 486-MS UV detector. The HPLC column used is a Nucleosil 100 5-C18 (250×4 mm, Macherey Nagel). The solvent A used is water containing 0.1% trifluoroacetic acid (TFA), the solvent B is acetonitrile containing 0.1% TFA. The flow rate is fixed at 1 ml/min. The elution is carried out in isocratic mode (10% A, 90% B) or with the aid of a linear gradient of 90% A/10% B to 10% A/90% B in the course of 25 minutes followed by 5 minutes with 10% A/90% B. A post-column for derivation allows the mixture to be directed to the mass spectrometers at a flow rate of 0.1 ml/min. The latter function with an electrospray fixed at 4 kV. The mass spectra are recorded between 100 and 800 Da in positive mode.

GC/MS (Gas Chromatography/Mass Spectrometry)

Analyses carried out with the aid of an HP 5890 CG gas chromatograph combined with a Finnigan MAT 8430 mass spectrometer. The silica capillary used is a J&W Sci DB-5 (30 m×0.32 mm, 0.25 μm film thickness). The gas used is helium under a pressure of 150 kPa. The temperature program is 60° C. (1 min), 30° C./min to 270° C. and then 10° C./minute to 320° C. The injector is heated to 250° C. The mass spectra are obtained in EI mode at 70 eV from 20 to 800 Da. The samples are injected before and after trimethylsilyl derivatization carried out with the aid of a mixture of pyridine and BSTFA (⅓, v/v) at 100° C. for 1 h.

NMR (−Nuclear Magnetic Resonance)

The spectra are obtained with the aid of a Brucker DPX-360 spectrometer at ambient temperature. Proton frequency 360.12 MHz, ¹³C frequency 90.56 MHz. Techniques applied for the proton NMR: one-dimension spectroscopy, 2D homonuclear correlation spectroscopy, 2D Overhauser nuclear spectroscopy. For the ¹³C NMR: one-dimension spectroscopy with and without proton decoupling, 2D heteronuclear correlation spectroscopy with detection of the ¹³C frequency. The molecules are dissolved in CDCl₃ 99.8%.

### EXAMPLES

### Example 1

Preparation of an Extract From Pleurotus eryngii

5 grams of the dried fruit of Pleurotus eryngii are ground and steeped in 50 ml 80% methanol for 1 day at ambient temperature. The mixture is then filtered over paper and the filtrate is collected and evaporated. The crude extract is taken up in distilled water (50 ml) and the pH is adjusted to 3 with the aid of 2N hydrochloric acid. This aqueous extract is extracted twice with ethyl acetate, volume for volume. The organic phase is dried with anhydrous Na₂SO₄ and evaporated in vacuo at 30° C. to remove the solvent. The extract obtained in this way, about 100 mg, is taken up in 4 ml methanol.

### Example 2

Preparation of an Extract from Pleurotus eous

The same procedure as that described in example 1 is used, with the difference that about 170 mg of extract are finally obtained from 4.5 g of dried fungus.

### Example 3

Preparation of an Extract from Ganoderma lucidum

The same procedure as that described in Example 1 is used, with the difference that 6 g of fungus are steeped and that about 140 mg of extract are finally obtained.

### Example 4

Preparation of an Extract From Grifola frondosa

The same procedure as that described in Example 1 is used, with the difference that 6.8 g of fungus are steeped and that about 180 g of extract are finally obtained.

### Example 5

Preparation of an Extract from Pleurotus ostreatus

The same procedure as that described in Example 1 is used, with the difference that about 120 mg of extract are finally obtained from 4 g of dried fungus.

### Example 6

Preparation of an Extract From Agrocybe aegerita

The same procedure as that described in Example 1 is used, with the difference that 6.5 g of fungus are steeped and that about 290 mg of extract are finally obtained.

### Example 7

Preparation of an Extract From Pholiota nameko

The same procedure as that described in Example 1 is used, with the difference that about 310 mg of extract are finally obtained from 4 g of dried fungus.

### Example 8

Preparation of an Extract From Pleurotus citrinopileatus

The same procedure as that described in Example 1 is used, with the difference that 7 g of fungus are steeped and that about 160 mg of extract are finally obtained.

### Example 9

Preparation of an Extract from Flamulina velutipes

The same procedure as that described in Example 1 is used, with the difference that 2.6 g of fungus are steeped and that about 140 mg of extract are finally obtained.

### Example 10

Fractionation and Physico-Chemical Characterization of Compounds Contained in the Extracts of the Fungus Ganoderma lucidum

These were chosen since the crude extracts obtained have a high hypocholesterolaemic activity in vitro.

200 g of the dry fungus Ganoderma lucidum are ground and steeped in 2 liters of 80% methanol at ambient temperature for 2 days. The mixture is filtered over gauze and the liquid phase is evaporated in vacuo at 30° C. The methanolic extract obtained (approx. 12 g) is taken up in 100 ml water and the pH of this solution is adjusted to 3 with the aid of 2N hydrochloric acid. This aqueous extract is extracted 3 times, volume for volume, with ethyl acetate. The organic phase is dried with anhydrous Na₂SO₄ and evaporated in vacuo at 30° C. to remove the solvent. The dry extract obtained (6.7 g) is taken up with the aid of a mixture of petroleum ether and 90% methanol. The methanolic extract obtained (6.5 g) is chromatographed over a silica gel; the elution is carried out in stages with 100% CHCl₃, then 1% MeOH in CHCl₃, then 10% MeOH in CHCl₃ and then 100% MeOH. The 100% CHCl₃ fraction is rechromatographed over silica gel; the elution is carried out in stages with 100% hexane, then 5% ethyl acetate in hexane, then 20% ethyl acetate in hexane, then 50% ethyl acetate in hexane and then 100% ethyl acetate. The active fractions of 20% ethyl acetate in hexane, 50% ethyl acetate in hexane and 100% ethyl acetate are purified by high performance liquid chromatography (HPLC). For this purpose, a Nucleosil 100-5 C18 column (250×4 mm, Macherey Nagel) is used with a Lichrospher 100 RP-18 post-column (Merck). The mobile phase consists of a mixture of 0.05% H₃PO₄ in water/acetonitrile (10/90) v/v. The chromatographic elution is carried out in isocratic mode with a flow rate of 1 ml/min. The detector used is a Hewlett Packard G1315A, series 1100 and λmax is fixed at 254 nm.

The active molecules are identified by mass spectroscopy (MS) and nuclear magnetic resonance (NMR).

### Example 11

Fractionation and Physico-Chemical Characterization of Compounds Contained in the Extracts of the Fungus Pleurotus citrinopileatus

These were chosen since the crude extracts obtained have a high hypocholesterolaemic activity in vitro.

200 g of the dry fungus Pleurotus citrinopileatus are ground and steeped in 2 liters of 80% methanol at ambient temperature for 2 days. The mixture is filtered over gauze and the liquid phase is evaporated in vacuo at 30° C. The methanolic extract obtained (approx. 12 g) is taken up in 100 ml water and the pH of this solution is adjusted to 3 with the aid of 2N hydrochloric acid. This aqueous extract is extracted 3 times, volume for volume, with ethyl acetate. The organic phase is dried with anhydrous Na₂SO₄ and evaporated in vacuo at 30° C. to remove the solvent. The ethyl acetate extract (6.6 g) is chromatographed over a silica gel; the elution is carried out in stages with 100% CHCl₃, then 5% MeOH in CHCl₃, then 10% MeOH in CHCl₃ and then 100% MeOH. The 100% CHCl₃ fraction is rechromatographed over silica gel; the elution is carried out in stages with 100% hexane, then 5% ethyl acetate in hexane, then 20% ethyl acetate in hexane, then 50% ethyl acetate in hexane and then 100% ethyl acetate. The active fractions of 20% ethyl acetate in hexane, 50% ethyl acetate in hexane and 100% ethyl acetate are purified by high performance liquid chromatography (HPLC). For this purpose, a Nucleosil 100-5 C18 column (250×4 mm, Macherey Nagel) is used with a Lichrospher 100 RP-18 post-column (Merck). The mobile phase consists of a mixture of 0.05% H₃PO₄ in water/acetonitrile (10/90) v/v. The flow rate is 1 ml/min. The detector used is a Hewlett Packard G1315A, series 1100 and λ max is fixed at 254 nm.

The active molecules are identified by mass spectroscopy coupled with a gas phase chromatography GC/MS.

Table 3 shows the oxygenated natural derivatives of lanosterols (oxylanosterols) identified in various purified fractions from Ganoderma lucidum and from Pleurotus citrinopileatus. As regards Ganoderma lucidum, the mass spectrometry and nuclear magnetic resonance analyses were carried out with the fractions of 80% hexane/20% ethyl acetate and 50% hexane/50% ethyl acetate (as indicated in Table 2). As regards Pleurotus citrinopileatus, only the fraction of 80% hexane/20% ethyl acetate was used.

Table 2: In vitro hypocholesterolaemic activity observed on human hepatic cells with the purified fractions of extracts obtained from the edible fungi Ganoderma lucidum and Pleurotus citrinopileatus

| | |
|---|---|
| Fractions | ID₅₀ (μg/ml) |

Ganoderma lucidum

| | |
|---|---|
| 100% CHCl₃ | |
| 95% hexane—5% ethyl acetate | >17 |
| 80% hexane—20% ethyl acetate | 3 |
| 50% hexane—50% ethyl acetate | 0.8 |
| 100% ethyl acetate | 1.5 |
| 95% CHCl₃—5% MeOH | 1.3 |
| 90% CHCl₃—10% MeOH | 2.4 |
| 100% MeOH | >2 |

Pleurotus citrinopileatus

| | |
|---|---|
| 100% CHCl₃ | |
| 95% hexane—5% ethyl acetate | >16 |
| 80% hexane—20% ethyl acetate | 7.5 |
| 50% hexane—50% ethyl acetate | 5 |
| 100% ethyl acetate | 2.5 |
| 95% CHCl₃—5% MeOH | 15 |
| 90% CHCl₃—10% MeOH | >60 |
| 100% MeOH | >55 |

Table 3: In vitro hypocholesterolaemic activity observed on human hepatic cells with the main purified oxylanosterol molecules of extracts obtained from the edible fungi Ganoderma lucidum and Pleurotus citrinopileatus.

| Oxylanosterols | Mass (m/z) | ID₅₀ (μq/ml) |
|---|---|---|
| ^{a}Ganoderol A | 438 | 1 |
| ^{b}Ganoderal A | 436 | 7 |
| ^{b}Ganoderol B | 440 | 10 |
| ^{b}Ganoderic acid | 454 | 0.5 |
| Y | | |

^{a}molecule present in Ganoderma lucidum and Pleurotus citrinopileatus
^{b}Molecule present only in Ganoderma lucidum

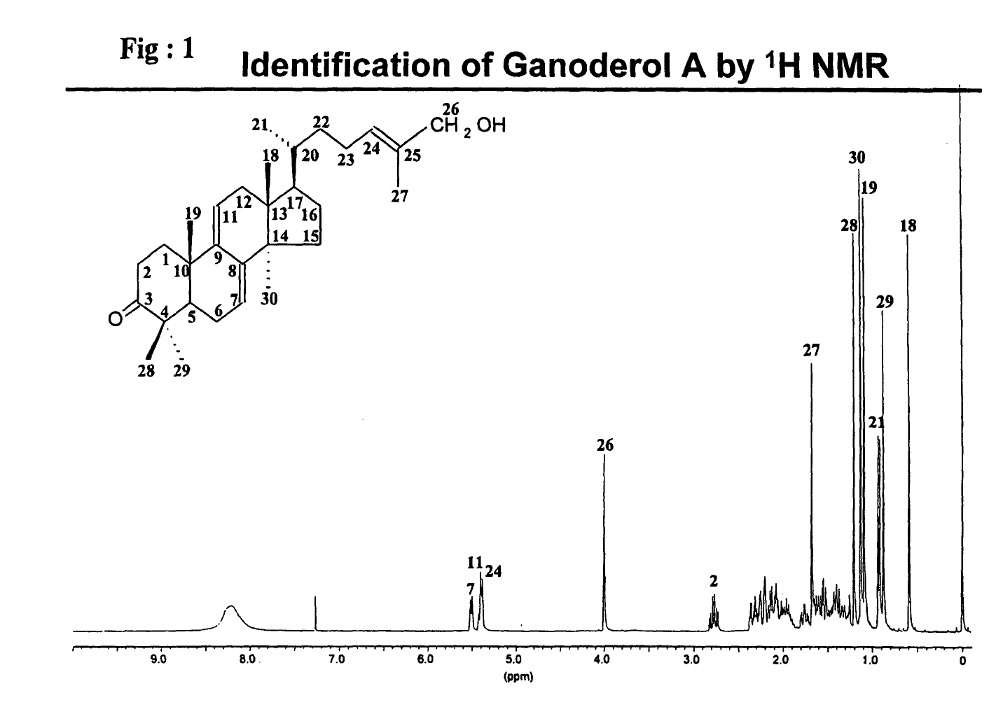

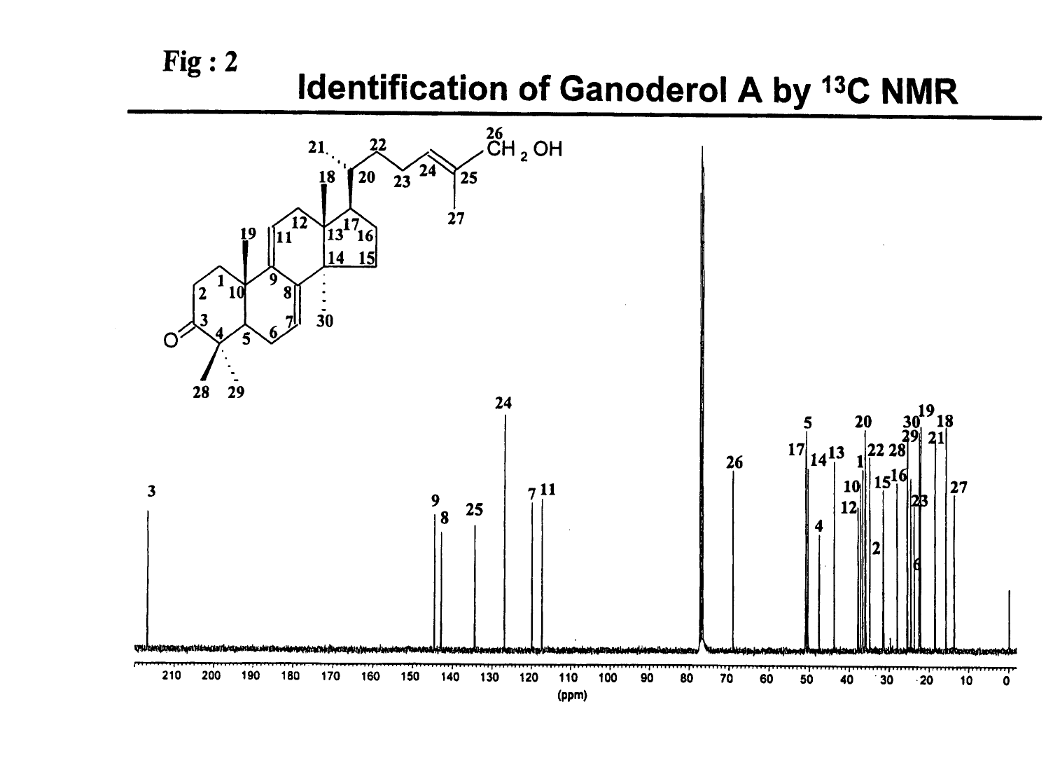

FIGS. 1 and 2 show the Ganoderol A identified by NMR and mass spectrometry in the purified fractions of Ganoderma lucidum (80/20 and 50/50 hexane/ethyl acetate) and of Pleurotus citrinopileatus (80/20 hexane/ethyl acetate).

What is claimed is:

1. A process for preparing a hypocholesterolaemic agent which comprises:
steeping an edible fungus in a first solvent under temperature and time conditions effective to extract an active fraction in a liquid phase,
separating the liquid phase from solid materials,
obtaining a dry extract of the active fraction from the liquid phase,
forming an aqueous phase of the dry extract and water,
contacting the aqueous phase with a second solvent that has a lower polarity than the first solvent and that is immiscible with water, with the contacting conducted under conditions sufficient to extract the active fraction in an organic phase,
separating the organic phase from the aqueous phase, and
obtaining as the hypocholesterolaemic agent the active fraction recovered from the organic phase.

2. The process of claim 1 wherein the fungi are Agaricalles, Aphyllophorales or Stereales.

3. The process of claim 1 wherein the fungi are one or more of Pleurotus eryngii, Pleurotus eous, Ganoderma lucidum, Grifola frondosa, Pleurotus ostreatus, Agrocybe aegerita, Pholiota nameko, Pleurotus citrinopileatus or Flamulina velutipes.

4. The process of claim 1 the steeping is carried out for 4 to 96 hours at a temperature of between 5 and 30° C.

5. The process of claim 1 wherein the dry extract is obtained by evaporating the liquid phase.

6. The process of claim 1 wherein the first solvent is methanol, ethanol, chloroform, or a mixture thereof.

7. The process of claim 1 wherein the second solvent is ethyl acetate, isopropanol, chloroform or a mixture thereof.

8. The process of claim 1 which further comprises adjusting the pH of the aqueous phase to a value of between 2 and 5 before the extraction with the second solvent.

9. The process of claim 1 wherein the contacting is carried out by repeated washings of the aqueous phase.

10. A hypocholesterolaemic agent obtainable by the process of claim 1 and being rich in oxygenated natural derivatives of lanosterol.

11. An edible composition for inhibiting synthesis of cholesterol in a person comprising a food or beverage and the hypocholesterolaemic agent of claim 11 in an effective amount therein.

12. A method for inhibiting synthesis of cholesterol which comprises administering to a person in need of such treatment the hypocholesterolaemic agent of claim 11 in an effective amount thereof.

13. A method for inhibiting synthesis of cholesterol which comprises administering to a person in need of such treatment a food or beverage containing the hypocholesterolaemic agent of claim 11 in an effective amount therein.
